(19) United States
(12) Patent Application Publication
(10) Pub. No.: US 2012/0330213 A1
(43) Pub. Date: Dec. 27, 2012

(54) Not an elastic slim wraps

(76) Inventor: Luis Oscar Valdez, Apopka, FL (US)

(21) Appl. No.: 13/506,934

(22) Filed: May 25, 2012

Related U.S. Application Data
(60) Provisional application No. 61/519,572, filed on May 25, 2011.

(51) Int. Cl. A61F 13/00

(52) U.S. Cl. 602/75

(57) ABSTRACT

The preceding objects and other characteristics and advantages are managed by an adapted wrap to cover a part of the human body. The not an elastic slim wrap made of a group of fabrics and plastic, to form shell. The slim wrap model contains portions, united by front Velcro's panels. These parts have the strength to wrap around a person's body. In addition for adjustment to size when the counter is reduced during treatment. It also facilitates in the terms of hygiene, by allowing the user to put it on and to take it off as they please. The not an elastic slim wrap can be complemented by other products used for the same purpose; massage creams, wet plaster therapy, etc. The product will be produce in a variety of sizes; Small to 6 XLarge.

### DESCRIPTION OF THE PREFFERED EMBODIMENTS

This application claims priority under 35 USC 119(e) based on provisional patent application No. 61/519,572 filed on May 25, 2011, which is herein incorporated by reference in its entirety.

### FIELD OF THE INVENTION

The present invention relates to a not an elastic slim wrap made of non elastic materials non stretchable material primarily to be under clothing and used passively such as during sleep or normal daily activities. It can reduce abdominal or waist fat.

### BACKGROUND ART

Use of abdominal compression is widely known in a number of distinct applications, with quite a long history. Further, the broad concept of a belt or band is that it is worn around a user's waist as a piece of clothing or as an exercise accessory is very old and well known.

One widely known use for abdominal compression, the long an extensive history of corsets warrants a mention. The corset is an interesting garment, which to most people in our modern world seems a very strange piece of underwear. The corset has at all times been used for shaping the body, most often for compressing the waist, but sometimes for raising the bust.

the abdominal compression discussion of girdles must note for the sake of parity that these body shaping garment are not limited to women. Girdle type garments have been recently marketed to men.

Advertisement for male girdles became popular In the 1930s but many of the products struggled to look different than women's undergarments. Whether for men or women that girdle is primarily a body shaping garment that as a result does not insolate or work the abdominal muscles for exercise and toning.

various types of slim wraps and bands have been used by people to slim their waist. Generally, such wraps are comprised simply of cylinder elastic band fitted to the waist. It has been noted that such elastic wraps quickly stretch, making them less useful by reducing the ability to burn the fat. Furthermore, the fact that the traditional elastic wraps to become thin because they are constructed of elastic materials causing the muscles to become more grown. The invention wrap is constructed of non elastic material that helps to eliminate the fat.

U.S. Pat. No. 3,189,920—Countour wrap-around pencil slim skit; 1965 Kurzyniec. U.S. Pat. No. 0015190A1—Preparation for reducing and/or preventing body fat and receptive uses in particular together with a dressing material; 2010 Saliwnachik U.S. 2004 0194189 a1—Fitness war; 2004 Lin. U.S. 2002 0082537 A1—Hip and shoulder wrap; 2002 MacAllister. U.S. 2010 0275339 A1—body cover wrap; 2010 Hue. U.S. 00556017 1A—Chiropractic BRAACE; 1996 Muncy.

### SUMMARY OF THE INVENTION

The preceding objects and other characteristics and advantages are managed by an adapted wrap to cover a part of the human body. The not an elastic slim wrap is made of a group of fabrics and plastic, to form shell. The slim wrap model contains portions, united by front Velcro's panels. These parts have the strength to wrap around a person's body. In addition to adjusting to size when the counter is reduced during treatment. It also facilitates in the terms of hygiene, by allowing the user to put it on and to take it off as they please. The not an elastic slim wrap can be complemented by other products used for the same purpose; massage creams, wet plaster therapy, etc. The product will be produce in a variety of sizes; Small to 6 XLarge.

is to provide not an elastic slim wrap adjust to the contour or forms of the body in the opportunity for waist.

is to provide a discharge burst of strength, which the consumer can re-use, with the waist slim wrap.

is to provide an economical treatment to the consumers.

is to provide a waist wrap that is simple to wear, and does not require the help of a second person.

is to provide a wrap in respect be flexible with the user's time.

is to provide a slim wrap with the facility to adjust to the physiological structure and user's hygiene.

is to provide slim wraps of very good visual appearance which motivates the user to wear it.

is to provide slim wraps with flexibility in the proportion relation to plastic and hardness.

is to provide the user with different models that fill the user's needs during their rest, during sleep and /or during work, etc.

### BRIEF DESCRIPTION OF THE DRAWINGS

FIG. 1—is a front and top view of the waist slim wrap 8

FIG. 2—is a right side view of the waist slim wrap 8.

FIG. 3—is a back side view of the waist slim wrap 8.

FIG. 4—The left side view of the waist slim wrap 8.

FIG. 5—Shows the three layers those components: c) is the outer layers of Denim fabric 100% cotton; d) shows the middle layer that is composed of is the plastic, wire and the support bands. e) Is the inner layer of towel fabric. Also it shows in the e) is a transversal cut x-y view to show the fabrics positions folded and type of stitches of the waist slim wrap 8

FIG. 6—Shows from open to embodiment wrap 8.

### DESCRIPTION OF THE PREFERRED EMBODIMENTS

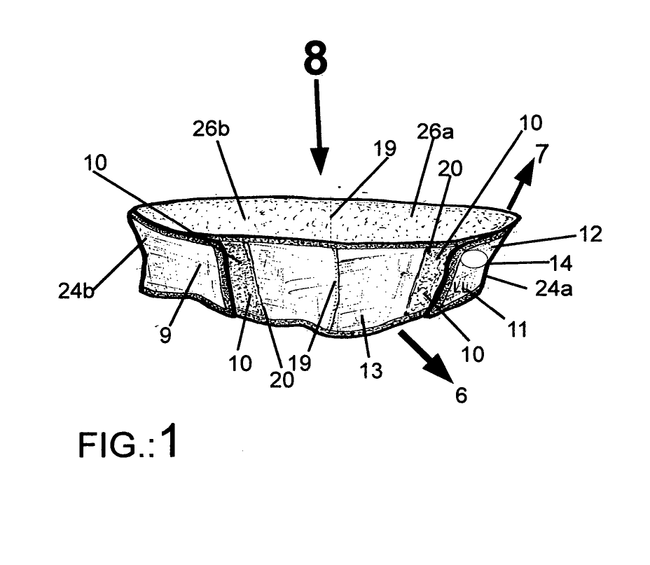

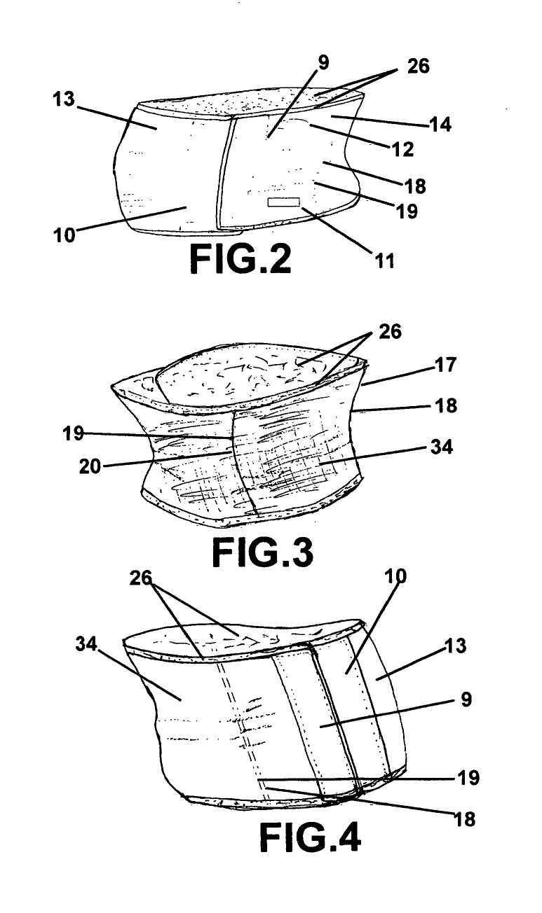

FIG. 1—Shows in the front and top views of not elastic waist slim wrap shaped 8 shows the Velcro 9's hook, stitched with single needle stitch, the loops Velcro 2′ and 4″ with number 10. Each band of loops Velcro 10 is used to adjust the wrap during the use. The care label 11 put by heat fabric transfer directly to the material. The trade mark 12 putted by heat fabric transfer process. Front Panel 13. Right band panel 14 that is supporting the hook Velcro 9; care label 11 and trademark 12. This panel is joined by over lock stitch inside 19 and top stitch in one needle stitch 20. Also shows the slim wrap's back panels 26a and 26b. Side panels 24a and 24b. Front Panel 6; Waist Band 7; is the Slim wraps name 8. Inside closed stitch with 2 needles over lock stitch machine 17. Top stitch in 1 needle machine (18) of FIG. 2.

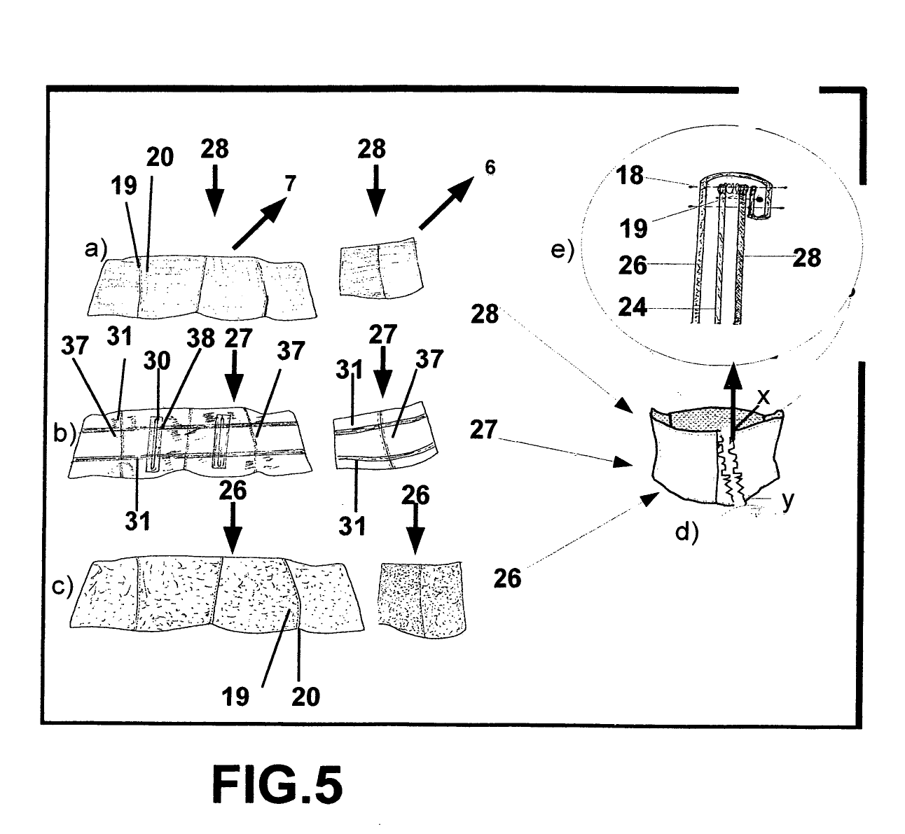

Towel fabric 100% cotton 26 of the FIG. 5. Transparent plastic #16, 18 , or 20. In the 27. Cotton 100% fabric denim or other similar fabric 28. Wrap wire (stainless steel) plastic cover 30. Support Tape 31. Sealed plastic or sewn 37.38-wire cover of plastic or other fabric material of the figure.

FIG. 2—Shows the right sides view of a waist slim wrap 8. Care label 11, trademark 12, loops Velcro 10. The material join 19, each fabric is joined separately by 2 needles over lock stitch and later with a single needle re-stitch 18, stitches to separate it.

FIG. 3—Shows a back side of a band 7 views of a not elastic waist slim wrap 8. It is formed by two panels joined by over lock stitch 19 and later stitched by top stitch 1 needle 18. Also it shows the towel fabric 26 of all contours inside of slim wrap 8.

FIG. 4—Shows a left side view of a waist slim wrap. It shows side panel 14, left loops Velcro 10. Towel fabric 26. Back panel left side 34. The panel's material joined 19; each fabric is joined separately by 2 needles over lock (SA/ST) stitch 19 and later top stitch 18 separately.

FIG. 5—Shows the three layers that components: a) is the outer layers of Denim fabric 100% cotton; b) shows the middle layer that is composed of is the plastic, wire and the support bands. c) Is the inner layer of towel's fabric. Also it shows in the d) is a transversal cut x-y view to show the fabrics positions folded and type of stitches of the waist slim wrap 8 e) shows a better view of the cut x-y.

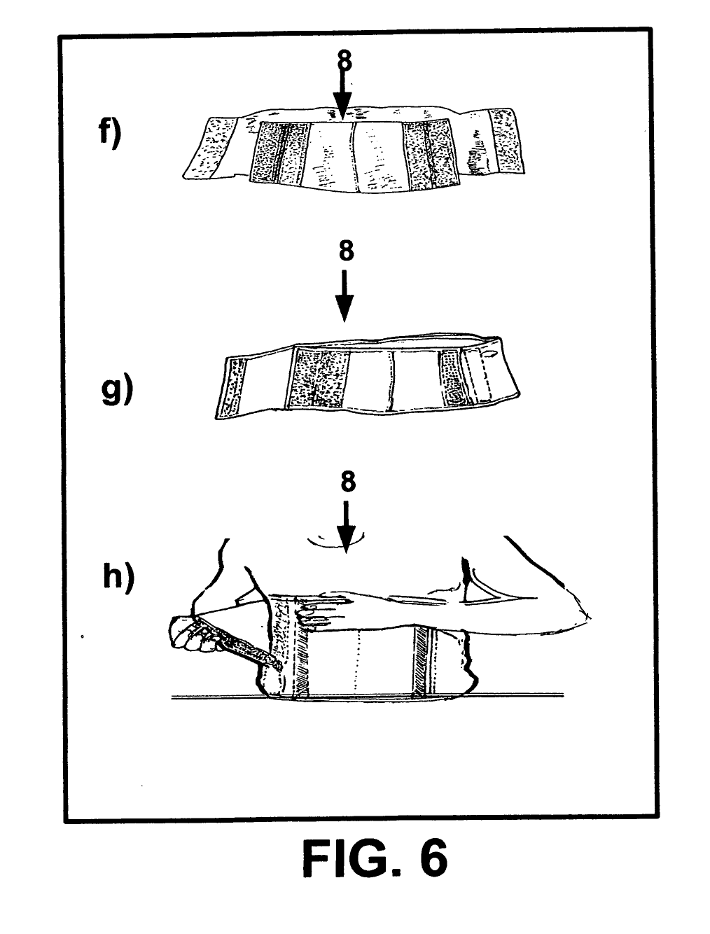

FIG. 6—Shows from open and closed the embodiment wrap 8. f) Shows the wraps 8 open on both sides, g) wrap 8 closed at left side and h) shows the wrap 8 embodiment to a human body.

### BRIEF DESCRIPTION OF THE DRAWINGS

FIG. 1—is a front and top view of the waist slim wrap 8

FIG. 2—is a right side view of the waist slim wrap 8.

FIG. 3—is a back side view of the waist slim wrap 8.

FIG. 4—The left side view of the waist slim wrap 8.

FIG. 5—Shows the three layers those components: c) is the outer layers of Denim fabric 100% cotton; d) shows the middle layer that is composed of is the plastic, wire and the support bands. e) Is the inner layer of towel fabric. Also it shows in the e) is a transversal cut x-y view to show the fabrics positions folded and type of stitches of the waist slim wrap 8

FIG. 6—Shows from open to embodiment wrap 8.

### DESCRIPTION OF THE PREFERRED EMBODIMENTS

FIG. 1—Shows in the front and top views of not elastic waist slim wrap shaped 8 shows the Velcro 9's hook, stitched with single needle stitch, the loops Velcro 2′ and 4″ with number 10. Each band of loops Velcro 10 is used to adjust the wrap during the use. The care label 11 put by heat fabric transfer directly to the material. The trade mark 12 putted by heat fabric transfer process. Front Panel 13. Right band panel 14 that is supporting the hook Velcro 9; care label 11 and trademark 12. This panel is joined by over lock stitch inside 19 and top stitch in one needle stitch 20. Also shows the slim wrap's back panels 26a and 26b. Side panels 24a and 24b. Front Panel 6; Waist Band 7; is the Slim wraps name 8. Inside closed stitch with 2 needles over lock stitch machine 17. Top stitch in 1 needle machine (18) of FIG. 2.

Towel fabric 100% cotton 26 of the FIG. 5. Transparent plastic #16, 18, or 20. In the 27. Cotton 100% fabric denim or other similar fabric 28. Wrap wire (stainless steel) plastic cover 30. Support Tape 31. Sealed plastic or sewn 37.38-wire cover of plastic or other fabric material of the figure.

FIG. 2—Shows the right sides view of a waist slim wrap 8. Care label 11, trademark 12, loops Velcro 10. The material join 19, each fabric is joined separately by 2 needles over lock stitch and later with a single needle re-stitch 18, stitches to separate it.

FIG. 3—Shows a back side of a band 7 views of a not elastic waist slim wrap 8. It is formed by two panels joined by over lock stitch 19 and later stitched by top stitch 1 needle 18. Also it shows the towel fabric 26 of all contours inside of slim wrap 8.

FIG. 4—Shows a left side view of a waist slim wrap. It shows side panel 14, left loops Velcro 10. Towel fabric 26. Back panel left side 34. The panel's material joined 19; each fabric is joined separately by 2 needles over lock (SA/ST) stitch 19 and later top stitch 18 separately.

FIG. 5—Shows the three layers that components: a) is the outer layers of Denim fabric 100% cotton; b) shows the middle layer that is composed of is the plastic, wire and the support bands. c) Is the inner layer of towel's fabric. Also it shows in the d) is a transversal cut x-y view to show the fabrics positions folded and type of stitches of the waist slim wrap 8 e) shows a better view of the cut x-y.

FIG. 6—Shows from open and closed the embodiment wrap 8. f) Shows the wraps 8 open on both sides, g) wrap 8 closed at left side and h) shows the wrap 8 embodiment to a human body.

1. This wrap is made of different layers of fabric and plastic; cut and sewn to form a wrap that molds the body when is used. Using the Velcro to in this wrap will constitute the release of fats and liquids of the abdomen.
A wrap to encircle the waist part of the body.
A waist slim wrap with the facility to put on an and take off by the right side or left side.
A wrap that controls the front hold by front panel's Velcro.
A Slim wrap that is cut and sewn to form the body contour.
A wrap with a separated front panel.
A wrap with Velcro of 2″ and 4″ at panel's sides.
A wrap with inside layer of Towel fabric 100% cotton.
A wrap with a layer of transparent plastic (#20, 14, or other for the band (7) and front panel (6).
A wrap with a layer of Denim or other 100% cotton fabric for the band (7)and panel (6) in exterior view.
A wrap with metal wire at the back side to put from 4″ to 7″ from the center of the back.
A wrap with inside 2 or 3 support tape for the front panel (6) and band (7).
A wrap with ⅜″ to ¾″ edge view around top and bottom of towel fabric 100% cotton.

2. The invention as defined in claim 1 where said wrap includes fabrics, plastic and fiber are interconnected thereto.

3. The invention as defined in claim 1 where said slim wrap is formed of non-elastic materials.

4. A wrap as described in claim 2 includes the following materials: Towel fabric 100% cotton, Transparent plastic #20 to #14.

5. The invention as defined in claim 1 wherein said fastening means includes a Velcro of 4″ and 2″, undergarment metal wrap wire or metal cable of ⅛″ plastic cover and silicone or rubber at wire's end point.

6. A slim wrap comprising of non-elastic materials in the abdomen wrap.

7. A wrap made with different length and width that encircle the abdomen, so it fits different sizes of people.
